(19) United States
(12) Patent Application Publication
(10) Pub. No.: US 2007/0010478 A1
(43) Pub. Date: Jan. 11, 2007

(54) Zosuquidar, daunorubicin, and cytarabine for the treatment of cancer

(76) Inventors: Branimir Sikic, Stanford, CA (US); Daniel Hoth, San Francisco, CA (US); David Socks, Carlsbad, CA (US); Scott Glenn, La Jolla, CA (US); John Marcelletti, San Diego, CA (US); Michael J. Walsh, San Diego, CA (US); Pratik S. Multani, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/416,829

(22) Filed: May 3, 2006

Related U.S. Application Data
(60) Provisional application No. 60/696,930, filed on Jul. 6, 2005.

(51) Int. Cl. A61K 31/7072; A61K 31/4709; A61K 31/704

(52) U.S. Cl. 514/49; 514/314; 514/34

(57) ABSTRACT

The present invention relates to a method of treating patients with solid tumors, leukemias, and other malignancies using a combination of zosuquidar, daunorubicin, and cytarabine. The invention is also directed to pharmaceutical formulations comprising zosuquidar, daunorubicin, and cytarabine. The formulations are particularly effective in treating relapsed Acute Myelogenous Leukemia (AML).

### RELATED APPLICATION

This application claims priority under 35 U.S.C. § 119(e) to U.S. Provisional Application No. 60/696,930 filed Jul. 6, 2005, which is incorporated by reference herein in its entirety, and which is hereby made a part of this specification.

### FIELD OF THE INVENTION

The present invention relates to a method of treating patients with solid tumors, leukemias, and other malignancies using a combination of zosuquidar, daunorubicin, and cytarabine. The invention is also directed to pharmaceutical formulations comprising zosuquidar, daunorubicin, and cytarabine. The formulations are particularly effective in treating relapsed Acute Myelogenous Leukemia (AML).

### BACKGROUND OF THE INVENTION

The field of oncology is in the midst of a major evolution. In the past, the treatment of cancer has been dominated by empiric, “one-size-fits-all” treatments based on types and stages of tumors. Toxic chemotherapy drugs have dominated the treatment landscape despite a very low cure rate, particularly against the most common cancers and those with known metastatic disease.

Now, treatments in development are targeted against specific proteins. Such targeting is based on a more robust knowledge of cancer mechanisms, which often crosses over many tumor types. These treatments are designed to work in defined subsets of patients, typically based on expression and function of the target protein rather than the type of tumor, and often in combination with standard chemotherapies. Advances in the molecular analysis of cancers will enable the identification of such susbsets of patients and the coupling of targeted therapeutics to novel diagnostic approaches.

The future of oncology lies in defining the disease in molecular terms (i.e., genetics, genomics, proteomics) and tailoring therapies according to individual tumor and normal cell properties. This new paradigm will predetermine likely responders, assess responses earlier, and adjust treatment based on continued molecular analyses of tumors.

Drug resistance is one of the most difficult problems that must be overcome in order to achieve successful treatment of human tumors with chemotherapy. Clinically, drug resistance, a characteristic of intrinsically resistant tumors (for example, colon, renal, and pancreas), may be evident at the onset of therapy. Alternatively, acquired drug resistance results when tumors initially respond to therapy but become refractory to subsequent treatments. Once a tumor has acquired resistance to a specific chemotherapeutic agent, it is common to observe collateral resistance to other structurally similar agents. The cellular mechanisms of drug resistance include apoptosis, drug uptake, DNA repair, altered drug targets, drug sequestration, detoxification, and efflux pumps (see, e.g., Dalton W. S. Semin. Oncol. 20:60, 1993).

Multidrug resistance (MDR), the ability of cancer cells to become resistant to the agent(s) actively used for therapy as well as other drugs that are structurally and functionally unrelated, is a particularly insidious form of drug resistance. This form of drug resistance is discussed in greater detail in Kuzmich et al., “Detoxification Mechanisms and Tumor Cell Resistance to Anticancer Drugs,” particularly section VII “The Multidrug-Resistant Phenotype (MDR),” Medical Research Reviews, Vol. 11, No. 2, 185-217, particularly 208-213 (1991); and in Georges et al., “Multidrug Resistance and Chemosensitization: Therapeutic Implications for Cancer Chemotherapy,” Advances in Pharmacology, Vol. 21, 185-220 (1990).

Although MDR may be caused by a variety of factors, one of the most prevalent forms of MDR is the type associated with overexpression of P-glycoprotein (P-gp). P-gp is a member of a superfamily of membrane proteins, termed adenosine triphosphate (ATP)-binding cassette (ABC) proteins, which behave as ATP-dependent transporters and/or ion channels for a wide variety of hydrophobic substrates. P-gp is a multiple transmembrane-spanning glycoprotein. Transfection experiments with the P-gp gene (mdr1) have conferred MDR to drug-sensitive tumor cells by providing an energy-dependent efflux pump that lowers the intracellular concentration of the cytotoxic agent, thereby allowing survival of the cell.

P-gp is expressed in normal biliary canaliculi of the liver, the adrenal cortex and proximal tubules of the kidney, and intestinal epithelia including the columnar cells of the large and small intestines; capillary endothelial cells of brain, testis, and placenta; and in the hematopoietic stem cells of bone marrow. It possesses excretory, protective, and barrier functions. P-gp is constitutively expressed or selected in many human cancers, and confers resistance to therapeutic agents including anthracyclines (e.g., doxorubicin, daunorubicin, epirubicin, idarubicin, mitoxantrone), vincas (e.g., vincristine, vinblastine, vinorelbine, vindesine), Topoisomerase-II inhibitors (e.g., etoposide, teniposide), taxanes (e.g., paclitaxel, docetaxel), and others (e.g., Gleevec, Mylotarg, dactinomycin, mithramycin).

The relative promiscuity of drug transport by P-gp and other MDR-associated transporters inspired a wide search for compounds that would not be cytotoxic themselves but would inhibit MDR transport. The initial demonstration of verapamil as a P-gp inhibitor was followed by many additional compounds reported to inhibit drug transport and thus sensitize MDR cells to chemotherapeutic drugs. Variously called chemosensitizers, MDR reversal agents, modulators, or converters, these ‘first generation’ MDR drugs included compounds of diverse structure and function such as calcium channel blockers (e.g., verapamil), immunosuppressants (e.g., cyclosporin A), antibiotics (e.g., erythromycin), antimalarials (e.g., quinine), and others (e.g., biricodar, tariquidar, valspodar).

First generation MDR drugs were not specifically developed for inhibiting MDR. They often had other pharmacological activities, as well as a relatively low affinity for MDR transporters and thus were limited in application. For example, P-gp has a low affinity for verapamil, thus requiring cardiotoxic levels for full modulator activity. In spite of the fact that only low serum levels could be obtained in a Phase II trial, 5 of 22 patients responded to a combination of verapamil and VAD (vincristine, doxorubicin, and dexamethasone). Four of the responders had elevated P-gp expression and function. Thus, verapamil has demonstrated some clinical utility in overcoming drug resistance. Cyclosporin A alters the pharmacokinetics of coadministered cytotoxic agents, resulting in significantly increased exposure to the oncolytic, thus confounding the interpretation of clinical trials.

Further characterization of the P-gp pharmacophore led to the identification of ‘second generation’ modulators based on the first generation but specifically selected or designed to reduce the side effects of the latter by eliminating their non-MDR pharmacological actions. Compounds such as the R-enantiomers of verapamil (R-verapamil) and dexniguldipine did not fare any better as MDR drugs in clinical studies, most likely because their affinity towards P-gp still fell short of producing significant inhibition of MDR in vivo at tolerable doses.

A more promising second generation modulator with a higher affinity towards P-gp was valspodar, a non-immunosuppressive cyclosporin D derivative. While early trials were encouraging, further work revealed significant pharmacokinetic interactions with several anticancer drugs. Although discontinued by Novartis, valspodar was studied in a Phase III study in elderly patients with acute myelogenous leukemia. Enrollment in the valspodar arm was halted due to excessive early mortality, most likely due to the PK interactions. Although the number of patients was limited, patients in the control arm whose pretreatment cells exhibited valspodar-modulated dye efflux in vitro (n=22) had worse outcomes than those without efflux (n=11) (complete remission, nonresponse, and death rates of 41%, 41%, and 18%, compared with 91%, 9%, and 0%; P=0.03), but with valspodar outcomes were nearly identical (Baer 2002). Moreover, for patients with valspodar-modulated efflux, median disease-free survival was 5 months in the control arm and 14 months with valspodar (P=0.07).

A second generation MDR modulator with activity against both P-gp and MRP1 (another ABC transporter associated with multidrug resistance) was biricodar. Vertex studied the agent in multiple Phase II studies of soft tissue sarcomas, ovarian cancer, small cell lung cancer, and others. However, biricodar and valspodar are both substrates for the P450 isoenzyme 3A4. Competition between cytotoxic agents and the P-gp inhibitors for cytochrome P450 3A4 resulted in unpredictable PK interactions and resulted in increased serum concentrations of cytoxics and, therefore, greater toxicity to the patient. A common response of clinical researchers has been to reduce the dose of the cytotoxic agents. However, the PK interactions are unpredictable and cannot be determined in advance. As a result, cytotoxic serum levels were either too high resulting in excessive toxicity or too low resulting in decreased efficacy. In addition to inhibiting P-gp, many of the second generation modulators function as substrates for other transporters, particularly the ABC family, inhibition of which could lessen the ability of normal, healthy cells to protect themselves from the cytotoxic agents.

### SUMMARY OF THE INVENTION

Dosage forms and treatment regimens for patients with solid tumors, leukemias, and other malignancies that result in increased rates of complete remission and increased cancer free survival rates are desirable. Particularly desirable are dosage forms and treatment regimens for AML patents that result in increased rates of complete remission and increased leukemia free survival and overall survival rates in newly diagnosed AML patients are desirable. The combined use of a P-gp inhibitor such as zosuquidar and chemotherapeutic agents such as daunorubicin and cytarabine enhances the therapeutic activity of the chemotherapeutics and can offer such advantages in the treatment of solid tumors, leukemias, and other malignancies.

Accordingly, in a first aspect a method of treating a malignancy is provided, the method comprising administering to a patient in need thereof zosuquidar, daunorubicin, and cytarabine.

In an embodiment of the first aspect, the malignancy is acute myelogenous leukemia.

In an embodiment of the first aspect, the malignancy is newly diagnosed acute myelogenous leukemia.

In an embodiment of the first aspect, the step of administering to a patient in need thereof zosuquidar, daunorubicin, and cytarabine comprises the steps of administering zosuquidar intravenously to a patient in an amount of from about 300 mg to about 800 mg administered continuously over from about 6 hours to about 24 hours on about 3 days; administering daunorubicin intravenously to a patient at a rate of from about 20 mg/m²/day to about 100 mg/m²/day for about 3 days, wherein administering daunorubicin is initiated from about 1 hour to about 5 hours after initiating administering zosuquidar; and administering cytarabine intravenously to a patient in an amount of from about 50 mg/m²/day to about 150 mg/m²/day continuously for about 7 days.

In an embodiment of the first aspect, the step of administering to a patient in need thereof zosuquidar, daunorubicin, and cytarabine comprises the steps of administering zosuquidar intravenously to a patient in an amount of from about 500 mg to about 700 mg administered continuously over from about 6 hours to about 24 hours on about 3 days; administering daunorubicin intravenously to a patient at a rate of from about 40 mg/m²/day to about 50 mg/m²/day for about 3 days, wherein administering daunorubicin is initiated from about 1 hour to about 4 hours after initiating administering zosuquidar; and administering cytarabine intravenously to a patient in an amount of from about 90 mg/m²/day to about 110 mg/m²/day continuously for about 7 days.

In an embodiment of the first aspect, the step of administering to a patient in need thereof zosuquidar, daunorubicin, and cytarabine comprises the steps of administering zosuquidar intravenously to a patient in an amount of from about 500 mg to about 700 mg administered continuously over from about 6 hours to about 24 hours on about 3 days; administering daunorubicin intravenously to a patient at a rate of about 45 mg/m²/day for about 3 days, wherein administering daunorubicin is initiated from about 1 hour to about 4 hours after initiating administering zosuquidar; and administering cytarabine intravenously to a patient in an amount of about 100 mg/m²/day continuously for about 7 days.

In an embodiment of the first aspect, the step of administering to a patient in need thereof zosuquidar, daunorubicin, and cytarabine comprises the steps of administering zosuquidar intravenously to a patient in an amount of from about 500 mg to about 700 mg administered continuously over from about 6 hours to about 24 hours on about 3 days; administering daunorubicin intravenously to a patient at a rate of about 45 mg/m²/day for about 3 days, wherein administering daunorubicin is initiated from about 1 hour to about 4 hours after initiating administering zosuquidar; and administering cytarabine intravenously to a patient in an amount of about 100 mg/m²/day continuously for about 7 days.

In an embodiment of the first aspect, the step of administering to a patient in need thereof zosuquidar, daunorubicin, and cytarabine comprises the steps of administering zosuquidar intravenously to a patient in an amount of from about 500 mg to about 700 mg administered continuously over about 6 hours on about 3 days; administering daunorubicin intravenously to a patient at a rate of about 45 mg/m²/day for about 3 days, wherein administering daunorubicin is initiated from about 1 hour to about 4 hours after initiating administering zosuquidar; and administering cytarabine intravenously to a patient in an amount of about 100 mg/m² day continuously for about 7 days.

In an embodiment of the first aspect, the step of administering to a patient in need thereof zosuquidar, daunorubicin, and cytarabine comprises the steps of administering zosuquidar intravenously to a patient in an amount of from about 550 mg administered continuously over about 6 hours on about 3 days; administering daunorubicin intravenously to a patient at a rate of about 45 mg/m²/day for about 3 days, wherein administering daunorubicin is initiated about 1 hour after initiating administering zosuquidar; and administering cytarabine intravenously to a patient in an amount of about 100 mg/m²/day continuously for about 7 days.

In a second aspect, a pharmaceutical kit for use in the treatment of relapsed acute myelogenous leukemia is provided, the kit comprising at least one dose of zosuquidar; directions for conducting at least one diagnostic for determining whether a patient exhibits at least one of positive efflux pump activity and positive P-gp expression or function; and directions for administering the zosuquidar in combination with a daunorubicin and cytarabine to treat newly dosed acute myelogenous leukemia in a patient exhibiting at least one of positive efflux pump activity and positive P-gp expression or function.

In a third aspect, a method of treating a malignancy in a patient is provided, the method comprising the steps of conducting a diagnostic test, whereby it is determined that the malignancy expresses or selects P-glycoprotein; and administering zosuquidar, daunorubicin, and cytarabine to the patient.

In an embodiment of the third aspect, the malignancy is acute myelogenous leukemia.

In an embodiment of the third aspect, the malignancy is newly diagnosed acute myelogenous leukemia.

In an embodiment of the third aspect, the malignancy is a carcinoma (e.g., breast cancer or ovarian cancer), a sarcoma, or a hematologic malignancy (e.g., acute lymphoblastic leukemia, chronic myeloid leukemia, plasma cell dyscrasias, lymphoma, or myelodysplasia).

In a fourth aspect, a method of treating a malignancy in a patient is provided, the method comprising the steps of conducting a diagnostic test, whereby it is determined that the malignancy exhibits positive efflux pump activity; and administering zosuquidar, daunorubicin, and cytarabine to the patient.

In an embodiment of the fourth aspect, the malignancy is acute myelogenous leukemia.

In an embodiment of the fourth aspect, the malignancy is newly diagnosed acute myelogenous leukemia.

In an embodiment of the fourth aspect, the malignancy is a carcinoma (e.g., breast cancer or ovarian cancer), a sarcoma, or a hematologic malignancy (e.g., acute lymphoblastic leukemia, chronic myeloid leukemia, plasma cell dyscrasias, lymphoma, or myelodysplasia).

In a fifth aspect, a method of treating a malignancy in a patient is provided, the method comprising the steps of conducting a diagnostic test, whereby it is determined that the malignancy expresses or selects P-glycoprotein or exhibits positive efflux pump activity; and administering zosuquidar and a chemotherapeutic agent that is a substrate for P-glycoprotein to the patient.

In an embodiment of the fifth aspect, the malignancy is acute myelogenous leukemia.

In an embodiment of the fifth aspect, the malignancy is newly diagnosed acute myelogenous leukemia.

In an embodiment of the fifth aspect, the malignancy is a carcinoma (e.g., breast cancer or ovarian cancer), a sarcoma, or a hematologic malignancy (e.g., acute lymphoblastic leukemia, chronic myeloid leukemia, plasma cell dyscrasias, lymphoma, or myelodysplasia).

In an embodiment of the fifth aspect, the chemotherapeutic agent is an anthracycline (e.g., doxorubicin, daunorubicin, epirubicin, idarubicin, or mitoxantrone).

In an embodiment of the fifth aspect, the chemotherapeutic agent is a Topoisomerase-II inhibitor (e.g., etoposide or teniposide).

In an embodiment of the fifth aspect, the chemotherapeutic agent is a vinca (e.g., vincristine, vinblastine, vinorelbine, or vindesine).

In an embodiment of the fifth aspect, the chemotherapeutic agent is a taxane (e.g., paclitaxel or docetaxel).

In an embodiment of the fifth aspect, the chemotherapeutic agent is Gleevec, dactinomycin, mitomycin, mithramycin, or Mylotarg.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

Cancer Targets

Many forms of cancer express P-gp, and thus can benefit from the administration of a P-gp efflux pump inhibitor when treated with a chemotherapeutic agent that is a substrate for P-gp efflux. For example, most solid tumors, lymphomas, bladder cancer, pancreatic cancer, ovarian cancer, liver cancer, myeloma, and sarcoma are all cancers with a P-gp expression of greater than 50%. Lymphocytic leukemia also has a P-gp expression of greater than 50%. The P-gp expression of breast cancers is about 30%. For metastatic breast cancer, 63% express P-gp. The methods and formulations of preferred embodiments are particularly efficacious in the treatment of any malignancy exhibiting some degree of P-gp expression or function, or in patients who are P-gp positive.

One form of cancer characterized by high rates of P-gp expression and function is acute myelogenous leukemia. There are approximately 11,000 new cases of AML per year in the United States and 9,000 new cases in the five major EU countries. In addition, the World Health Organization defines advanced myelodysplastic syndrome (MDS) as AML. There are approximately 4,000 cases of advanced MDS in the US and 3,000 cases in the five major EU countries. As a result, the target patient population for zosuquidar is approximately 15,000 patients in the U.S. and 12,000 in the major European markets.

Adult AML presents greater treatment challenges when compared to pediatric AML (age <15 years). Due in part to a more resilient patient population and a more sensitive disease, the 5 year survival rates for pediatric AML is 50% (late 1990s). In contrast, due in part to multiple co-morbid conditions and a more resistant disease, the 5 year survival rates for adult AML are only 13% (late 1990s). The 5 year survival rate for patients over 65 is only 7%.

Approximately 75% of AML patients are over age 60, and 71% are P-gp positive. Clinical outcomes in terms of patient survival rates are significantly better for patients that are P-gp negative than for those that are P-gp positive—a 50% survival rate at approximately 3-4 months for P-gp positive patients, versus a 50% survival rate at approximately 15 months for P-gp negative patients. See Campos, et al., Blood, 79:473-476, 1992.

Standard induction therapy in the U.S. for newly diagnosed, or de novo, AML patients is cytarabine with either idarubicin or daunorubicin (both P-gp substrates). In one study, 71% of AML patients greater than 60 years of age expressed moderate to high levels of P-gp. The expression was associated with a reduction in the complete remission (CR) rate. The CR rate for P-gp negative AML patients was 67% compared to 34% for P-gp positive patients. This combination of high levels of P-gp expression with the nearly universal use of drugs that are P-gp substrates provides a ready opportunity for the coadministration of a P-gp inhibitor in patients with AML.

Zosuquidar

U.S. Pat. Nos. 5,643,909 and 5,654,304 disclose a series of 10,11-methanobenzosuberane derivatives useful in enhancing the efficacy of existing cancer chemotherapeutics and for treating multidrug resistance. One such derivative having good activity, oral bioavailability, and stability, is zosuquidar, a compound of formula (2R)-anti-5-3-[4-(10,11-difluoromethanodibenzosuber-5-yl)piperazin-1-yl]-2-hydroxypropoxy)quinoline.

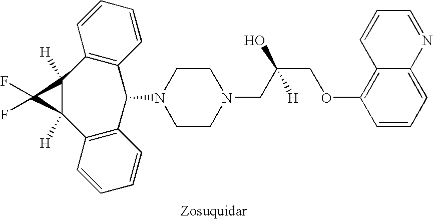

Given the limitations of previous generations of MDR modulators, three preclinical critical success factors were identified and met for zosuquidar: 1) it is a potent inhibitor of P-glycoprotein; 2) it is selective for P-glycoprotein; and 3) no pharmacokinetic interaction with co-administered chemotherapy is observed.

Zosuquidar is extremely potent in vitro (Kᵢ=59 nM) and is among the most active modulators of P-gp-associated resistance described to date. Zosuquidar has also demonstrated good in vivo activity in preclinical animal studies. In addition, the compound does not appear to be a substrate for P-gp efflux, resulting in a relatively long duration of reversal activity in resistant cells even after the modulator has been withdrawn.

Another significant attribute of zosuquidar as an MDR modulator is the minimal pharmacokinetic (PK) interactions with several oncolytics tested in preclinical models. Such minimal PK interaction permits normal doses of oncolytics to be administered and also a more straightforward interpretation of the clinical results.

Daunorubicin

Daunorubicin is an antibiotic chemotherapy treatment that is widely used to treat acute myeloid leukemia and acute lymphocytic leukemia. It is produced by the bacteria Streptomyces coeruleorubidis and was approved by the FDA as a first line therapy treatment for leukemia in 1998. Daunorubicin is typically administered intravenously. It is marketed under the brand names Cerubidine, DaunoXome, and Liposomal daunorubicin. Daunorubicin has the following structure: 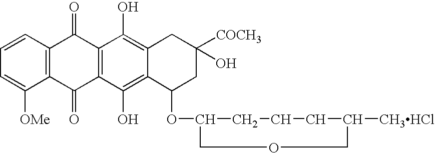
Cytarabine

Cytarabine is a deoxycytidine analogue, cytosine arabinoside (ara-C), which is metabolically activated to the triphosphate nucleotide (ara-CTP), which acts as a competitive inhibitor of DNA polymerase and produces S phase-specific cytotoxicity. It is used as an antineoplastic, generally as part of a combination chemotherapy regimen, in the treatment of acute lymphocytic and acute myelogenous leukemia, the blast phase of chronic myelogenous leukemia, erythroleukemia, and non-Hodgkin's lymphoma. It is typically administered intravenously and subcutaneously, and for the prophylaxis and treatment of meningeal leukemia, administered intrathecally. Cytarabine has the following structure: 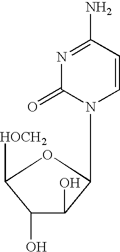
Chemotherapeutic Regimens Utilizing Zosuquidar Daunorubicin, and Cytarabine

The combination of zosuquidar, a highly specific and safe P-gp efflux inhibitor, in combination with the antibiotic chemotherapeutic daunorubicin and the antineoplastic cytarabine, is effective for treatment of leukemias, especially newly diagnosed AML. Likewise, the formulations and dosing regimens employing zosuquidar, daunorubicin, and cytarabine can be employed in treating AML patients other than newly diagnosed AML patients, or for treatment of other types of leukemia, lymphomas or lymphocytic leukemia. The effective dose of zosuquidar and the timing of administration of zosuquidar, daunorubicin, and cytarabine are critical to achieving improved complete remission rates and enhanced leukemia free survival rates in the newly diagnosed AML patient population.

While the methods and formulations of preferred embodiments are especially preferred for treatment of newly diagnosed AML patients, the methods and formulations can be adapted to other drugs and indications. For example, zosuquidar, daunorubicin, and cytarabine can be administered according to the disclosed dosing regimens, or slightly modified dosing regimens, for treatment of other types of leukemia or other cancers that express P-gp and/or exhibit P-gp function, e.g., many solid tumors, bladder cancer, pancreatic cancer, liver cancer, myeloma, carcinomas (e.g., breast cancer and ovarian cancer), sarcomas, and hematologic malignancies other than AML (e.g., acute lymphoblastic leukemia, chronic myeloid leukemia, plasma cell dyscrasias, lymphoma, myelodysplasia).

Zosuquidar, daunorubicin, and cytarabine or certain other therapeutic agents can be administered in the form of a pharmaceutically acceptable salt, e.g., the trihydrochloride salt. The terms “pharmaceutically acceptable salts” and “a pharmaceutically acceptable salt thereof” as used herein are broad terms and are used in their ordinary sense, including, without limitation, to refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of zosuquidar, daunorubicin, or cytarabine or other therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives can also be suitable for use in compositions and methods of preferred embodiments. Also suitable for administration are selected therapeutic agents in hydrated form, selected enantiomeric forms of certain therapeutic agents, racemic mixtures of certain therapeutic agents, and the like.

Contemplated routes of administration include topical, oral, subcutaneous, parenteral, intradermal, intramuscular, intraperitoneal, and intravenous. However, it is particularly preferred to administer zosuquidar, daunorubicin, and/or cytarabine in intravenous form. The combination or individual components can be in any suitable solid or liquid form. A particularly preferred form comprises a lyophilized form that is reconstituted for intravenous administration.

Zosuquidar, daunorubicin, and/or cytarabine can be formulated into liquid preparations for, e.g., oral, nasal, anal, rectal, buccal, vaginal, peroral, intragastric, mucosal, perlingual, alveolar, gingival, olfactory, or respiratory mucosa administration. Suitable forms for such administration include suspensions, syrups, and elixirs. If nasal or respiratory (mucosal) administration is desired (e.g., aerosol inhalation or insufflation), compositions may be in a form and dispensed by a squeeze spray dispenser, pump dispenser or aerosol dispenser. Aerosols are usually under pressure by means of a hydrocarbon. Pump dispensers can preferably dispense a metered dose or a dose having a particular particle size.

The pharmaceutical compositions containing zosuquidar, daunorubicin, and/or cytarabine are preferably isotonic with the blood or other body fluid of the patient. The isotonicity of the compositions can be attained using sodium tartrate, propylene glycol or other inorganic or organic solutes. Sodium chloride is particularly preferred. Buffering agents can be employed, such as acetic acid and salts thereof, citric acid and salts thereof, boric acid and salts thereof, and phosphoric acid and salts thereof. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like.

Viscosity of the pharmaceutical compositions can be maintained at a selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener can depend upon the thickening agent selected. An amount is preferably used that can achieve the selected viscosity. Viscous compositions are normally prepared from solutions by the addition of such thickening agents.

A pharmaceutically acceptable preservative can be employed to increase the shelf life of the pharmaceutical compositions. Benzyl alcohol can be suitable, although a variety of preservatives including, for example, parabens, thimerosal, chlorobutanol, and benzalkonium chloride can also be employed. A suitable concentration of the preservative is typically from about 0.02% to about 2% based on the total weight of the composition, although larger or smaller amounts can be desirable depending upon the agent selected.

The zosuquidar, daunorubicin, and/or cytarabine can be in admixture with a suitable carrier, diluent, or excipient such as sterile water, physiological saline, glucose, and the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., standard texts such as “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the zosuquidar, daunorubicin, and/or cytarabine can be provided as a tablet, aqueous or oil suspension, dispersible powder or granule, emulsion, hard or soft capsule, syrup, or elixir. Compositions intended for oral administration can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

Formulations for oral administration can also be provided as hard gelatin capsules, wherein the zosuquidar, daunorubicin, and/or cytarabine are mixed with an inert solid diluent, such as calcium carbonate, calcium phosphate, or kaolin, or as soft gelatin capsules. In soft capsules, the active ingredients can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the zosuquidar, daunorubicin, and/or cytarabine in admixture with fillers such as lactose, binders such as starches, and/or lubricants such as talc and magnesium stearate and, optionally, stabilizers.

Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s) including zosuquidar, daunorubicin, and/or cytarabine, preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the zosuquidar, daunorubicin, and/or cytarabine in admixture with non-toxic pharmaceutically acceptable excipients including inert materials. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet or capsule contains from about 10 mg or less to about 1,000 mg or more of each of zosuquidar, daunorubicin, and/or cytarabine, more preferably from about 20, 30, 40, 50, 60, 70, 80, 90, or 100 mg to about 150, 200, 250, 300, 350, 400, 450, 500, 550, 600, 650, 700, 750, 800, or 900 mg. Most preferably, tablets or capsules are provided in a range of dosages to permit divided dosages to be administered. A dosage appropriate to the patient and the number of doses to be administered daily can thus be conveniently selected. While in certain embodiments it can be preferred to incorporate the zosuquidar, daunorubicin, cytarabine, and any other therapeutic agent employed in combination therewith in a single tablet or other dosage form, in certain embodiments it can be desirable to provide the zosuquidar, daunorubicin, cytarabine, and other therapeutic agents in separate dosage forms, e.g., each of zosuquidar, daunorubicin, and cytarabine in separate dosage forms, or daunorubicin and cytarabine in one dosage form and zosuquidar alone in another. Combinations of dosage forms can also be employed, e.g., oral and intravenous.

Suitable inert materials include diluents, such as carbohydrates, mannitol, lactose, anhydrous lactose, cellulose, sucrose, modified dextrans, starch, and the like, and inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya, and tragacanth, and alginic acid and salts thereof.

Binders can be used to form a hard tablet. Binders include materials from natural products such as acacia, tragacanth, starch, gelatin, methyl cellulose, ethyl cellulose, carboxymethyl cellulose, polyvinyl pyrrolidone, hydroxypropylmethyl cellulose, and the like.

Lubricants, such as stearic acid and magnesium or calcium salts thereof, polytetrafluoroethylene, liquid paraffin, vegetable oils, waxes, sodium lauryl sulfate, magnesium lauryl sulfate, polyethylene glycol, starch, talc, pyrogenic silica, hydrated silicoaluminate, and the like can be included in tablet formulations.

Surfactants can also be employed, for example, anionic detergents such as sodium lauryl sulfate, dioctyl sodium sulfosuccinate, and dioctyl sodium sulfonate, cationic detergents such as benzalkonium chloride and benzethonium chloride, and/or nonionic detergents such as polyoxyethylene hydrogenated castor oil, glycerol monostearate, polysorbates, sucrose fatty acid ester, methyl cellulose, and carboxymethyl cellulose.

Controlled-release formulations can be employed wherein the zosuquidar, daunorubicin, and/or cytarabine are incorporated into an inert matrix that permits release by either diffusion or leaching mechanisms. Slowly degenerating matrices can also be incorporated into the formulation. Other delivery systems can include timed release, delayed release, or sustained release delivery systems. Nanoparticulate systems or nanoparticulate forms of the active ingredients can advantageously be employed in certain embodiments.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone, polyethylene glycols, and enteric materials such as phthalic acid esters. Dyestuffs and pigments can be added for identification or to characterize different combinations of active compound doses

When administered orally in liquid form, a liquid carrier such as water, petroleum, oils of animal or plant origin such as peanut oil, mineral oil, soybean oil, or sesame oil, or synthetic oils can be added to the zosuquidar, daunorubicin, and/or cytarabine. Physiological saline solution, dextrose, other saccharide solutions, and glycols such as ethylene glycol, propylene glycol, and polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

Pulmonary delivery of zosuquidar, daunorubicin, and/or cytarabine can also be employed. The zosuquidar, daunorubicin, and/or cytarabine are delivered to the lungs while inhaling and traverses across the lung epithelial lining to the blood stream. A wide range of mechanical devices designed for pulmonary delivery of therapeutic products can be employed, including but not limited to nebulizers, metered dose inhalers, and powder inhalers, all of which are familiar to those skilled in the art. These devices employ formulations suitable for the dispensing of zosuquidar, daunorubicin, and/or cytarabine. Typically, each formulation is specific to the type of device employed and can involve the use of an appropriate propellant material, in addition to diluents, adjuvants, and/or carriers useful in therapy.

The zosuquidar, daunorubicin, cytarabine, and/or other optional active ingredients are advantageously prepared for pulmonary delivery in particulate form with an average particle size of from 0.1 μm or less to 10 μm or more, more preferably from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of zosuquidar, daunorubicin, and/or cytarabine include carbohydrates such as trehalose, mannitol, xylitoi, sucrose, lactose, and sorbitol. Other ingredients for use in formulations can include dipalmitoylphosphatidylcholine (DPPC), 1,2-sn-dioleoylphosphatidylcholine (DOPE), disteroylphosphatidylcholine (DSPC), and dioleoylphosphatidyl-choline (DOPC). Natural or synthetic surfactants can be used, including polyethylene glycol and dextrans, such as cyclodextran. Bile salts and other related enhancers, as well as cellulose and cellulose derivatives, and amino acids can also be used. Liposomes, microcapsules, microspheres, inclusion complexes, and other types of carriers can also be employed.

Pharmaceutical formulations suitable for use with a nebulizer, either jet or ultrasonic, typically comprise the zosuquidar, daunorubicin, and/or cytarabine dissolved or suspended in water at a concentration of about 0.01 mg or less to 100 mg or more of each of zosuquidar, daunorubicin, and/or cytarabine per mL of solution, preferably from about 0.1, 1, 2, 3, 4, 5, 6, 7, 8, 9, or 10 mg per mL of solution to about 15, 20, 25, 30, 35, 40, 45, 50, 55, 60, 65, 70, 75, 80, 85, or 90 mg per mL of solution. The formulation can also include a buffer and a simple sugar (e.g., for protein stabilization and regulation of osmotic pressure). The nebulizer formulation can also contain a surfactant to reduce or prevent surface induced aggregation of the zosuquidar, daunorubicin, and/or cytarabine caused by atomization of the solution in forming the aerosol.

Formulations for use with a metered-dose inhaler device generally comprise a finely divided powder containing the active ingredients suspended in a propellant with the aid of a surfactant. The propellant can include conventional propellants, such as chlorofluorocarbons, hydrochlorofluorocarbons, hydrofluorocarbons, and hydrocarbons. Preferred propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations suitable for dispensing from a powder inhaler device typically comprise a finely divided dry powder containing zosuquidar, daunorubicin, and/or cytarabine, optionally including a bulking agent, such as lactose, sorbitol, sucrose, mannitol, trehalose, or xylitol, in an amount that facilitates dispersal of the powder from the device, typically from about 1 wt. % or less to 99 wt. % or more of the formulation, preferably from about 5, 10, 15, 20, 25, 30, 35, 40, 45, or 50 wt. % to about 55, 60, 65, 70, 75, 80, 85, or 90 wt. % of the formulation.

When zosuquidar, daunorubicin, and/or cytarabine are administered by intravenous, cutaneous, subcutaneous, parenteral, or other injection, they are preferably in the form of pyrogen-free, parenterally acceptable aqueous solutions or oleaginous suspensions. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed, including synthetic monoglycerides and diglycerides. In addition, fatty acids such as oleic acid can likewise be used in the formation of injectable preparations. The pharmaceutical compositions can also contain stabilizers, preservatives, buffers, antioxidants, and other additives known to those of skill in the art.

The duration of the injection can be adjusted depending upon various factors, and can comprise a single injection administered over the course of a few seconds or less to 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 40, 44, 48, 54, 60, 66, 72, 78, 84, 90, or 96 hours or more of continuous intravenous administration. In some embodiments, the injection can be administered over the course of up to 5, 6, 7, 8, 9, 10, or more days.

The zosuquidar, daunorubicin, and/or cytarabine can be administered systemically or locally, via a liquid or gel, or as an implant or device.

The compositions of the preferred embodiments can additionally employ adjunct components conventionally found in pharmaceutical compositions in their art-established fashion and at their art-established levels. Thus, for example, the compositions can contain additional compatible pharmaceutically active materials for combination therapy (such as supplemental P-gp inhibitors, chemotherapeutic agents, and the like), or can contain materials useful in physically formulating various dosage forms of the preferred embodiments, such as excipients, dyes, perfumes, thickening agents, stabilizers, preservatives and antioxidants.

The zosuquidar, daunorubicin, and/or cytarabine can be provided to an administering physician or other health care professional in the form of a kit. The kit is a package which houses one or more containers which contain zosuquidar, daunorubicin, and/or cytarabine in suitable form and instructions for administering the pharmaceutical composition to a subject. The kit can optionally also contain one or more additional therapeutic agents. The kit can optionally contain one or more diagnostic tools and instructions for use, e.g., a diagnostic to measure efflux pump activity or P-gp expression or function. For example, a kit containing a single composition comprising zosuquidar, daunorubicin, and/or cytarabine in combination with one or more additional therapeutic agents can be provided, or separate pharmaceutical compositions containing zosuquidar, daunorubicin, and/or cytarabine, and additional therapeutic agents can be provided. The kit can also contain separate doses of zosuquidar, daunorubicin, and/or cytarabine for serial or sequential administration. The kit can contain suitable delivery devices, e.g., syringes, inhalation devices, and the like, along with instructions for administrating zosuquidar, daunorubicin, and/or cytarabine and any other therapeutic agent. The kit can optionally contain instructions for storage, reconstitution (if applicable), and administration of any or all therapeutic agents included. The kits can include a plurality of containers reflecting the number of administrations to be given to a subject.

In a particularly preferred embodiment, a kit for the treatment of AML, especially newly diagnosed AML, is provided that includes zosuquidar, daunorubicin, and cytarabine and instructions for administering each. In another particularly preferred embodiment, a kit for the treatment of newly diagnosed AML is provided that includes zosuquidar, daunorubicin, and/or cytarabine and one or more diagnostics or instructions for conducting one or more diagnostics for determining P-gp expression and/or efflux pump activity (function). The kit can also include instructions, an assay, and/or a diagnostic for determining if a patient has AML.

The combination of zosuquidar, daunorubicin, and cytarabine can be administered to a patient having a leukemia, a solid tumor, or other malignancy. It is particularly preferred to administer the combination when P-gp expression is positive, or to use the combination in the treatment of a malignancy exhibiting P-gp expression or function. Cancer targets exhibiting a P-gp expression >50% of patients are particularly preferred for treatment by the combinations of the preferred embodiments. Dosage regimes as described below for AML can also be suitable for the treatment of other leukemias, solid tumors, bladder cancer, pancreatic cancer, liver cancer, myeloma, carcinomas (e.g., breast cancer and ovarian cancer), sarcomas, and other hematologic malignancies (e.g., acute lymphoblastic leukemia, chronic myeloid leukemia, plasma cell dyscrasias, lymphoma, myelodysplasia).

Treatment of Acute Myelogenous Leukemia

The combination of zosuquidar, daunorubicin, and cytarabine are most preferably administered to newly diagnosed AML patients. However, the combination can also be administered prophylactically to patients believed to be suffering from AML prior to confirmation of the diagnosis, or to AML patients other than newly diagnosed AML patients (e.g., relapsed AML patients). The administration route, amount administered, and frequency of administration can vary depending on the age of the patient, status as relapsed or newly diagnosed AML patient, and severity of the condition.

Contemplated amounts of zosuquidar for intravenous administration to treat newly diagnosed AML are from about 400 mg/day or less to about 1,600 mg/day or more, preferably from about 500, 600, or 700 mg/day to about 900, 1000, 1100, 1200, 1300, 1400, or 1500 mg/day, and most preferably 700 mg/day. In the course of a treatment regimen, the zosuquidar is preferably administered on two, three, or four separate days. The dosage is preferably administered in intravenously continuously over the course of about 6 to about 90 hours, more preferably over the course of about 12, 18, 24, 30, 36, or 42 hours to about 54, 60, 66, 72, 78, or 84 hours, most preferably over about 24 hours, 48 hours, or 72 hours, depending upon the treatment regimen. Preferably the zosuquidar is administered on Day 1 of the treatment regimen. In certain embodiments, additional zosuquidar is administered on Day 2, on Days 2 and 3, or on Days 2, 15, and 16. However, in certain embodiments, one, two, or three or more additional doses can be administered on other days of the treatment regimen.

Contemplated amounts of daunorubicin for intravenous administration to treat newly diagnosed AML are from about 10 mg/m²/day or less to about 100 mg/m²/day or more administered at initiation of zosuquidar infusion or up to about 1, 2, 3, 4, 5, or 6 or more hours after initiation of zosuquidar infusion. The dosage is preferably administered intravenously at a rate of about 25 mg/m²/day or less to about 90 mg/m²/day or more, preferably about 30, 35, or 40 mg/m²/day or less to about 50, 55, 60, 65, 70, 75, 80, or 85 mg/m²/day, and most preferably about 45 mg/m²/day continuously over the course of about 2 or 2.5 days to about 3.5 or 4 days, preferably about 3 days.

Contemplated amounts of cytarabine for intravenous administration to treat newly diagnosed AML are from about 10 mg/day or less to about 3,000 mg/day or more administered at initiation of zosuquidar infusion or after initiation of zosuquidar infusion. The dosage is preferably administered intravenously at a rate of about 50 mg/m²/day or less to about 200 mg/m²/day or more, preferably 60, 70, 80, or 90 mg/m²/day or less to about 110, 120, 130, 140, 150, 160, 170, 180, or 190 mg/m²/day, and most preferably about 100 mg/m²/day continuously over the course of about 1, 2, 3, 4, 5, or 6 days up to about 8, 9, or 10 days or more, preferably over about 7 days.

A particularly preferred dosing regimen for newly diagnosed AML includes continuous intravenous administration of 550 mg of zosuquidar over 6 hours (3 days), continuous intravenous administration of cytarabine at a rate of 100 mg/m²/day (7 days), and intravenous administration of daunorubicin at a dose of 45 mg/m²/day (3 days), wherein infusion of daunorubicin is started 1 hour after initiation of zosuquidar infusion. Another particularly preferred dosing regimen includes continuous intravenous administration (preferably about 1 to 24 hours in duration, more preferably about 6 to 24 hours in duration, most preferably about 24 hours in duration) of 500 to 700 mg/day of zosuquidar (3 days), continuous intravenous administration of cytarabine at a rate of 100 mg/m²/day (7 days), and intravenous administration of daunorubicin at a dose of 45 mg/m²/day (3 days), wherein infusion of daunorubicin is started 1 to 4 hours after initiation of zosuquidar infusion. While in the above described embodiments infusion of daunorubicin is started after a specified time period has lapsed after initiation of zosuquidar infusion, in other embodiments other start times can be preferred, e.g., immediately after or during initiation of zosuquidar infusion up to about 1, 2, 3, 4, 5, 6, 7, 8, 9, 10, 11, 12, or more hours after initiation of zosuquidar infusion.

The above-described dosing regimens for treatment of newly diagnosed AML can also be adapted to the treatment of relapsed AML as well as metastatic breast cancer or other carcinomas.

While the above methods of the preferred embodiments have been discussed primarily in connection with the treatment of AML, the methods are also particularly efficacious when daunorubicin, a P-gp substrate, is used to treat other malignancies exhibiting some degree of P-gp expression.

Experiments

Patients with AML were treated with zosuquidar (700 mg/day continuous intravenous infusion) for 72 hours beginning on day 1, and 4 hour before the start of therapy with daunorubicin (45 mg/m² intravenous on days 1, 2, and 3). Cytarabine was also administered (100 mg/m²/day) starting after the first dose of daunorubicin as a continuous intravenous infusion on days 1-7. Blood samples were taken at intervals for pharmacokinetic and pharmacodynamic studies. Pharmacokinetic drug determinations were conducted by HPLC. Pharmacodynamic assessments of cellular P-gp function were conducted using an accumulation assay with DiOC2 and flow cytometry. P-gp function was assessed on circulating natural killer (NK) cells and leukemic blasts. FIG. 1 presents the relationships between plasma zosuquidar levels and inhibition of P-gp function. Means of 3 patients for each time point are shown. Peak zosuquidar levels were achieved by 24 hours post-initiation of drug infusion. The levels of zosuquidar remained relative stable at 180-207 ng/ml for the remainder of the infusion period. After the 72 hour time point when zosuquidar infusion had been halted, plasma zosuquidar levels rapidly decreased to approximately 50-60 ng/ml at the 80-96 hour time point.

P-gp function for both NK cells and leukemic blasts was potently inhibited within 2 hours after the start of zosuquidar infusion. Inhibition of P-gp function can be attributed to zosuquidar since treatment with daunorubicin and cytarabine were initiated after the 4 hour time point. Inhibition of P-gp functional activity was maintained throughout the infusion period, and continued for at least 12 hours after zosuquidar infusion was halted. These results indicate that it is possible give a relatively short infusion of zosuquidar allowing for continued inhibition of leukemia cell P-gp function after the infusion has been halted and lessening the occurrence of adverse events such as central nervous system toxicities.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A method of treating a malignancy in a patient, the method comprising the steps of:
conducting a diagnostic test, whereby it is determined that the malignancy expresses or selects P-glycoprotein; and
administering zosuquidar, daunorubicin, and cytarabine to the patient.

2. The method of claim 1, wherein the malignancy is acute myelogenous leukemia.

3. The method of claim 1, wherein the malignancy is newly diagnosed acute myelogenous leukemia.

4. The method of claim 1, wherein the malignancy is a carcinoma.

5. The method of claim 4, wherein the carcinoma is breast cancer.

6. The method of claim 4, wherein the carcinoma is ovarian cancer.

7. The method of claim 1, wherein the malignancy is a sarcoma.

8. The method of claim 1, wherein the malignancy is a hematologic malignancy selected from the group consisting of acute lymphoblastic leukemia, chronic myeloid leukemia, plasma cell dyscrasias, lymphoma, and myelodysplasia.

9. A method of treating a malignancy in a patient, the method comprising the steps of:
conducting a diagnostic test, whereby it is determined that the malignancy exhibits positive efflux pump activity; and
administering zosuquidar, daunorubicin, and cytarabine to the patient.

10. The method of claim 9, wherein the malignancy is acute myelogenous leukemia.

11. The method of claim 9, wherein the malignancy is newly diagnosed acute myelogenous leukemia.

12. The method of claim 9, wherein the malignancy is a carcinoma.

13. The method of claim 12, wherein the carcinoma is breast cancer.

14. The method of claim 12, wherein the carcinoma is ovarian cancer.

15. The method of claim 9, wherein the malignancy is a sarcoma.

16. The method of claim 9, wherein the malignancy is a hematologic malignancy selected from the group consisting of acute lymphoblastic leukemia, chronic myeloid leukemia, plasma cell dyscrasias, lymphoma, and myelodysplasia.
